(19) United States
(12) Patent Application Publication
(10) Pub. No.: US 2011/0159104 A1
(43) Pub. Date: Jun. 30, 2011

(54) MICROEMULSION

(76) Inventor: Alexander Teslenko, Hagen (DE)

(21) Appl. No.: 13/054,734

(22) PCT Filed: Jul. 22, 2009
(86) PCT No.: PCT/EP09/05318

§ 371 Date: Mar. 16, 2011

(30) Foreign Application Priority Data
Jul. 26, 2008 (DE) 10-2008-034.944.5

(51) Int. Cl. A61K 35/64; A61P 29/00; A61P 23/02; A61P 31/00; A61P 31/10; A61P 31/12; A61P 37/06; A61P 37/02; A61P 17/06; A61P 17/12; A61P 3/02; A61P 39/06; A61P 39/04; A61P 17/00

(52) U.S. Cl. 424/537

(57) ABSTRACT

The present invention relates to biocompatible microemulsions based on honey which can additionally comprise both water-soluble and also fat-soluble active ingredients in stable form. Via the microemulsion bioavailable nutrients bonded in the honey, preferably together with further active substances, are introduced by topical application into the skin or orally, nasally or percutaneously into the body where they develop their positive effects. These emulsions can be prepared easily and are used both in medicine/veterinary medicine, dermatology and also in cosmetics. Moreover, the honey microemulsions can be used on their own or with nutritionally relevant substances in the fields of foodstuff, functional foods, food supplements or dietetic products.

Honey is a natural product and contains approximately 200 different ingredients. Depending on the type of honey the composition may vary greatly. The quantitatively most important ingredients are fructose (27 to 40%), glucose (22 to 41%) and water (approx. 18%). Additional typical ingredients are other types of sugar, pollen, mineral nutrients, proteins, enzymes, amino acids, vitamins, coloring to matter and flavoring substances. Honey may have a liquid or solid (crystallized) consistency. This depends mainly on the ratio of the two monosaccharides fructose and glucose present in the honey but also on the way honey is processed and stored.

Since the Stone Age honey has been used by man and for a very long time has is been the only sweetening agent available. Later, emerging processes that led to the production of inexpensive granulated sugar (pure saccharose) from sugar beet and cane resulted in honey becoming to a large extent superseded in this respect. Nevertheless, honey continues to be valued as aliment, for example as sweet spread on bread or as an alternative to industrially produced granulated sugar. Nowadays, due to its positive and health-promoting effects honey experiences a renaissance. Many of the substances contained in honey are not only of significance nutritionally but also have positive effects on the health of men and animals.

Even many thousand years ago honey was used as a medicament also in naturopathy in addition to other bee products. More recently, an especially processed germ-free honey (so-called medical honey) is also used for the therapy or tending of wounds. A number of honey products that have been granted approval as medicinal products in the form of gels, wound dressings or pads for the healing of wounds can be found on the market in many countries.

However, honey left in its natural state must not be used for the treatment of wounds. Although germs are incapable of reproducing in honey it cannot be ruled out for certain that they exist in natural honey in the form of impurities. For that reason, honey employed for medical purposes is subjected to sterilization by gamma ray treatment before it is put to use. Other than a thermal sterilization approach this treatment will not destroy the enzymes indispensable for the healing process.

Due to the fact that an increasing number of germs have become resistant to antibiotics the bactericidal and bacteriostatic effects of honey are very interesting, primarily in the medical field. Even a 10-40% honey kills gram-negative bacteria resistant to antibiotics after two days of application (P. H. Kwakmann et al., Clin Infect. Dis. 46 (11), 1677-82 (2008); P. J. Taormina et al., 2001, Int. J. Food Microbiol. 69 (3), 217-25 (2001)).

Nowadays, hydrogen peroxide (H₂O₂) is considered the most important antibacterial active agent present in honey. Hydrogen peroxide and gluconic acid are produced with the aid of glucose oxidase during the oxidation of water and glucose. Antagonistic to the glucose oxidase is the catalase. Moreover, the formation of H₂O₂ is also influenced by heat and light which both have detrimental effects on glucose oxidase and thus impede the production of H₂O₂.

Aside from this, research in recent time has also focused on further ingredients (so-called inhibines) which have bactericidal, positive effects, said ingredients killing among others methicillin-resistant staphylococci and vancomycin-resistant enterococci. The non-peroxide substances (inhibines) present in honey stem primarily from plants. Four groups of substances found in honey have to a varying degree bacteriostatic effects as follows: They are of volatile, neutral, basic and acidulous nature. Among the inhibines count a number of vegetable substances which exhibit antibacterial activity: Terpenes, benzyl alcohol, pinocembrin, methyl syringates, hydroxy benzoic acid and others.

The very strong antibiotic effects of Manuka honey are ascribed to methylglyoxal which is found in this honey at an extraordinarily high concentration (often 1000 times).

Hitherto, only very few of the healing effects of honey have been investigated in detail in vitro, in vivo and in clinical studies. Substantial proof, however, exists with respect to the healing effects of honey in connection with topical applications.

Honey has slightly anti-inflammatory properties so that swellings, elevated temperatures and local pain recede. Honey is conducive to the growth of fibroblasts to causing wounds to heal in a more uniform manner with scars forming to a lesser extent (WO 2007/009185).

Honey is used as wound dressing due to the fact that it acts as slight antiseptic and also decomposes dead tissue existing in wounds. Meanwhile, three antimicrobial mechanisms of action are known, i.e. the very high osmolarity, hydrogen is peroxide and inhibines.

For the topical tending of wounds honey is applied in various pharmaceutical forms. A composition in the form of an ointment consisting of honey, olive oil and beeswax has shown favorable results in the topical treatment of hemorrhoids/piles and anal fissures. Also known are porous and non-porous wound dressings impregnated with honey (WO 2007/137881).

Positive effects are achieved with honey and honey preparations in the healing of wounds in the gastrointestinal tract, inter alia in the healing of peptic ulcers caused by Helicobacter pylori.

Honey preparations have been tried successfully for the healing of hemorrhoids/piles (U.S. Pat. No. 6,482,442).

In Russia medical products on honey basis are approved for use in dentistry, inter alia for the treatment of parodontosis.

Fungal infections of the skin surface count among the most frequently occurring diseases of the skin. They are usually caused by fungi (dermatophytes, yeast or mold) that are facultatively pathogenic to humans. Approximately 80% of all skin surface diseases originate from the effects of dermatophytes.

More than 90% of the prescriptions given by dermatologists and general practitioners relate to topical dermatotherapeutics. Aside from antimicrobially effective substances (among others antibiotics) used for the treatment of fungal skin infections keratolytic active agents, retinoids, benzoyl peroxide or acelainic acid are as well used in the treatment.

The antimycotic properties of honey are well known, among other things to provide assistance after chemotherapy and therapeutic radiology.

Also for the treatment of “problem skin” honey preparations have shown positive effects, e.g. in the treatment of psoriasis or tinea dermatoses.

A drawback of the above described honey preparations is that they are mainly applied in the form of an ointment, a cream, or gel or by means of impregnated cloth or wound dressings. Formulations applied in this way have only shown very low rates of penetration through the skin/mucous membrane.

Moreover, honey and honey preparations also have brought about positive effects (inter alia, systemic effects) when applied orally.

With healthy persons oxidative and antioxidative processes are found to be in equilibrium. Should this equilibrium be changed in favor of the oxidative processes the resultant effects are known as oxidative stress.

Aging processes and many diseases such as Alzheimer's disease, cancer, rheumatism, arteriosclerosis and diabetes mellitus are increasingly associated with the occurrence of oxidative stress.

The cardiovascular risk parameter identifies the probability to contract a cardiovascular disease such as cardiac infarction, apoplexia or peripheral arterial occlusion (PAO). This threat even increases in the presence of certain cardiovascular risk factors such as hypertonia, diabetes mellitus or dislipoproteinaemia.

The antioxidative effects of honey are primarily due to the presence of certain amino acids and catalase and as such honey may reduce cardiovascular risks.

In comparison to saccharose honey has brought about a significant reduction of cholesterol, LDL, triglycerides, C-reactive protein.

In bioassays with rats a honey diet was tested and showed a significantly lower increase in weight of the rats in relation to animals fed on a saccharose diet. Biochemical metabolism parameters (blood glucose level, triglyceride, cholesterol etc.) were also found to be significantly better with a honey diet.

The anti-inflammatory effects of honey and propolis have been examined in vivo and in vitro.

A formulation of honey and plant extracts (Semecarpus anacardium and Emblica officinalis) proved to be efficient with rheumatoid arthritis (RA) in rats thanks to its highly antioxidative effect and synergistically acting polyphenols, flavonoids and tannins. This honey formulation also showed analgesic and anti-pyretic effects comparable to diclofenac.

Traditionally, honey is used as bread spread all over the world.

Moreover, honey is an ingredient in bakery products, beverages, chocolate candy and numerous other food products and tests are being conducted to use it as natural preservation agent. Little is known about using honey for dietetic products, functional food and dietary supplements.

Honey may also be used for cosmetic applications. It is known that from the age of 20 onwards the skin already becomes significantly thinner also due to the lack of nutritional substrates caused by a degeneration of the papillary capillaries, so it starts aging and thus loses vitality. Therefore, to maintain the “vitality” of the skin cells, especially the cells of the stratum germinativum, an efficient vehicle must be provided via which the necessary cellular nutrition can be supplied in sufficient quantity to the cells from the outside. Cellular substrate predominantly are sugar, amino acids, secondary nutrients, minerals and oxygen. Aging and degenerative skin demonstrably shows lack of substrate and oxygen.

The most important cellular substrate is glucose of which sufficient amounts can presumably not always be supplied endogenously via the degenerated papillary loops. The availability of other sugar types such as, for example, glucosamine biosynthesized in the body from glucose also reduces greatly with age and these should be furnished as well.

Amino acids constitute a second important substrate necessary to support the synthesis performance of the cells.

To enable nutrients to be supplied to the cells oxygen is required as a third important substrate. Moreover, oxygen should be capable of reaching these cells easily via the facilitated diffusion mechanism.

Through a compensatory natural cell alimentation and by increasing the moisture of the skin efficiently the extracellular matrix can be repaired biologically by rebuilding glucosaminoglycans. Supplying the essential saccharides is a very important factor for the biosynthesis of glucosaminoglycans. Of special significance is also to improve the synthesis of collagen which can only be achieved when the bio-availability of ascorbic acid and oxygen can be ensured to an adequate extent. Improving the collagen synthesis results in the skin becoming tauter and smoother.

Most important in this context is to get through the barrier formed by the corneal layer.

Therefore, improving the penetration of the active substance into the skin is most significant. However, a systemic intake is not desirable in this case, for example with locally acting antimycotics or cosmetic preparations. In such cases the active substance may be used in a concentration that does not cause water-rich microemulsions to become supersaturated.

Honey can only absorb a limited amount and number of water-soluble substances without losing its above described properties.

In the presence of fat-soluble substances it will not be possible to produce a homogeneous honey solution. When using emulsifiers only coarse emulsions can be produced which are incapable of penetrating into the skin or mucous membrane.

To bring out the beneficial effects of honey for medical (wound healing, anti-inflammatory, cholesterol-lowering purposes etc.), cosmetic and dermocosmetic applications as well as in the foodstuff field new honey preparations should possess favorable skin and mucous membrane penetrating characteristics.

For both medical and cosmetic treatments penetration and permeation through the stratum corneum down to the vital cells of the epidermis are of great significance. A distinction must be made in this context between cosmetic skin care and cosmetic treatment.

Other than in cosmetic skin care a cosmetic treatment, especially in the frame-work of an anti-aging concept, involves supporting the vital cells with essential nutrients and thus enhances the restoration of the poorly functioning constituents of the skin, e.g. promotion of the collagen and hyaluronic acid biosynthesis, antioxidative or hormonal effects which are to be viewed as intracutaneous treatments.

The penetration effect of traditional cosmetic preparations such as ointments, creams, lotions, gels, masks and liposomes into the skin is negligible. Such preparations only penetrate into the upper layers of the stratum corneum (SC) and do not have any effect on the living cells of the SC, the epidermis and dermis.

Since the 80s the application of liposomes has been well known both in the medical and cosmetic field. Liposomes possess an envelope consisting of one or several water-phospholipid liquid crystalline double layers. They have an aqueous inner space used as container for water-soluble active substances, and they exist in an aqueous solution. The production of liposomes requires the use of specialized equipment and is very expensive. The penetration of liposomes into the stratum corneum is only poor.

It is thus the object of the present invention to provide honey formulations that have favorable skin/mucous membrane penetration characteristics and in this manner effectively bring active agents/substances to the relevant target location.

According to the invention this objective is reached by a microemulsion with
50 to 80% w/w of an oil phase,
2 to 40% w/w of a mixture of one or several W/O emulsifiers and one or several O/W emulsifiers at a ratio of 1:5 to 1:1,
5 to 40% w/w of honey, royal jelly, propolis and/or perga,
0.01 to 30% w/w of co-emulsifiers and
1 to 20% w/w of water or an aqueous solution.

Both water-soluble and fat-soluble active agents or pharmaceutical substances may at the same time be incorporated in the honey microemulsion without causing instability of the emulsion. Depending on water and/or fat solubility the active substances are part of the continuous or dispersed phase.

It has been found that the inventive microemulsions on the basis of honey or the bees products royal jelly, propolis and perga penetrate very quickly and deeply into the skin through the stratum corneum so that the skin will be enriched with the above mentioned substances. In this way the skin can be moisturized and skin smoothness improved. Furthermore, the microemulsions are capable of penetrating into mucous membranes so that positive or desirable effects are also brought about in the gastrointestinal tract, nose, ears, mouth or genitals.

Honey or the bee products royal jelly, propolis or perga introduced by way of the microemulsion into the skin/mucous membrane can thus produce in addition to moisture regulation further favorable effects for the skin. Its antioxidative properties take effect by way of the amino acids and catalases it contains. Further favorable properties include anti-inflammatory, antibiotic and immunity stimulating effects. With the help of the microemulsion pharmaceutical and cosmetic active substances can be introduced into the skin/mucous membrane.

The microemulsions may be used both in cosmetic and dermatological products to be applied to the skin/mucous membrane and in products intended for oral administration. These may be medical products as well as veterinary preparations and, moreover, food, dietary supplements or dietetic products.

It is also possible to provide microemulsions for spray application. Conceivable are microemulsions of liquid, sprayable, jelly-like, semi-solid and solid consistency.

Using microemulsions in foodstuff, dietary supplements and dietetic products offers a number of advantages. The microemulsion permits sensitive proteins such as enzymes to be protected against digestion and allows its solubilization and also improves the adsorption of active substances by the mucous membrane of the gastro-intestinal tract and enables antioxidative effects of oil and water-soluble vitamins and antioxidants.

The microemulsion according to the invention is a water-in-oil microemulsion (W/O microemulsion). This emulsion may, however, be changed into a secondary or oil-in-water microemulsion (O/W microemulsion) by converting it with a water phase (water or water/solvent mixture). O/W microemulsions are especially suited, inter alia, for spray application.

Preferably, a microemulsion contains 0 to 30% w/w, in particular 0.01 to 20% w/w of one or several water-soluble or fat-soluble active agents or pharmaceutical preparations.

Surprisingly, it was found that a primary W/O honey microemulsion could be prepared on the basis of customary oils, emulsifiers and co-emulsifiers, with synthetic, semi-synthetic and natural oils being suitable for the production of such primary W/O honey microemulsions. Both non-ionic and zwitterionic emulsifiers can be put to use. Phospholipids are in particular suited for use as co-emulsifiers.

Surfactant-containing multicomponent systems that are thermodynamically stable and have a transparent to slightly opalescent appearance are termed microemulsions. Such systems consist of an aqueous and an oil component as well as a surfactant and, as a rule, a cosurfactant which, being capable of loosening up the surfactant film, is meant to increase the interfacial fluidity. The cosurfactant penetrates between the surfactant molecules and causes the geometric packing of the surfactant layer and the geometric parameters of the droplets, resp. the packing parameters or their curvature to change. Depending on composition, water, oil or bicontinuous systems with a variety of structures (micelles, swollen micelles, structured micro areas, bicontinuous areas) may form.

Microemulsions possess the unique property in that the interfacial tension between the phases is very low. This results in the phases to be very thermodynamically stable against phase separation, and this means that e.g. the micelles are stable in the form of very small particles of a size ranging between 10 to 200 nm (other than in conventional emulsions with particle sizes of between 1 and 20 μm). As proposed by the present invention the diameter of the dispersed water droplets typically ranges between 20 and 150 nm.

It depends on the surfactant selected whether microemulsions of the O/W type (oil-in-water) or W/O type (water-in-oil) are formed. Information to this effect can be obtained, among other things, by checking the HLB value. If this value is found to be in the range of 4.0 and 7.0 W/O microemulsions are formed. Higher values ranging between 9.0 and 20.0 are indicative of O/W microemulsions.

Due to the fact that the oil and water concentrations of microemulsions may be equally high in a one-phase system, these emulsions have the ideal properties of a universal solvent. They may be administered both with lipophilic and hydrophilic substances.

Characterizing multi-component systems is a very difficult task. Microemulsions usually develop in a narrowly limited area within a so-called phase diagram. Even minor changes in composition may result in a displacement of the microemulsion areas within the phase diagram.

Microemulsions have a great variety of uses and can be administered in different forms. Microemulsions are prepared depending on the respective properties of the active substances and employed via various application routes (pharmaceutically, in cosmetics or in the food processing industries). In the field of veterinary medicine microemulsions enable pharmaceutical preparations to be produced for peroral or percutaneous applications in the treatment of inflammations, mastitis, gastroenteritis, and for the production of insecticides.

For cosmetic applications formulations with bioregulating activating substances (inter alia improving blood circulation and oxygen supply etc.) can be provided that enhance the cell growth (cellulite, anti-aging, hypoxia). Furthermore, formulations can be prepared that contain bioregulating inhibiting substances suppressing the growth of cells (antibacterial effects, combating skin aging, constrictive effects, anti-proliferative effects, for the treatment of psoriasis, tumors etc.). Among others, there are the following applications:
Anti-aging
Treatment of problem skin (acne, cellulite, psoriasis, photodermatosis)
Moisturizing
Bleaching
Coloring
Hair care
Treatment of alopecia
Nail care
Lipsticks
Soap

Irrespective of a concrete application field the inventive microemulsions inter alia offer the following advantages:
excellent solving capacity for hydrophilic and hydrophobic substances
high (thermodynamic) stability
ease of production
preservation agents may be waived
wide range of possible applications
easier penetration through skin and mucous membrane

### Oil Phase

Advantageously, the oil phase comes from the group of oils acceptable pharmaceutically and/or for use with food. For example, the oils may be esters of alkan carbonic acids and alcohols. Such ester oils may advantageously be selected from groups consisting of: Isopropyl myristate, isopropyl palmitate, isopropyl stearate, isopropyl oleate, n-butyl stearate, n-hexyl laurate, n-decyl oleate, isooctyl stearate, isononyl stearate, isononyl isonanoate, 2-ethylhexyl palmitate, 2-ethylhexyl laurate, 2-hexyldecyl stearate, 2-octyl dodecyl palmitate, ethyl oleate, oleyl oleate, oleyl eructate, erucyl oleate as well as synthetic, semi-synthetic, and natural mixtures of such esters.

Moreover, the oil phase can be selected from the group of the dialkyl ethers, the group of the alcohols as well as fatty acid triglycerides, in particular the triglycerine esters of saturated and/or unsaturated alkaline carboxylic acids having a chain length of between 8 and 24, particularly 12 to 18 C atoms. The fatty acid triglycerides may, for example, stem from the group of synthetic, semi-synthetic and natural oils, e.g. olive oil, almond oil, avocado oil, sunflower oil, soy bean oil, groundnut oil, rapeseed oil, palm oil, coconut oil, palm kernel oil and the like.

The oil phase may also be based on pharmaceutically acceptable oils such as 2-ethylhexyl isostearate, octyldodecanol, isotridecyl isonanoate, isoeicosane, 2-ethylhexyl cocoate, caprylic-caprinic acid triglyceride, dicaprylylic ether, almond oil, avocado oil or olive oil.

Furthermore, the oil phase may be generated by means of low-volatile hydrocarbons such as paraffin oil, squalene or squalane. Fatty alcohols having 6 to 18 carbon atoms in straight chains as well as acids from the group of lauryl, palmitinic, myristinic, arachidonic acid, linolenic and linolic acids may also be put to use.

The inventive microemulsion advantageously contains essential fatty acids, in particular linolic and γ-linolenic acid, oil acid, eicosapentaene acid or its derivatives, borage oil, evening primrose oil, dogrose oil, Rosa rubignosa, Centalla or inophyllum.

### Emulsifiers

For the production of the primary W/O microemulsion various types of emulsifiers may be employed, for example from the group of the ethoxylated fatty alcohols having 8-18 carbon atoms in straight chains, in particular polyethylene glycol(2)stearyl ether (Steareth-2), Steareth-20, Oleth-3 or Oleth-10.

Moreover, emulsifiers may advantageously stem from the group of the sorbitan derivatives such as sorbitan monolaurate or sorbitan trioleate or from the group of the ethoxylated sorbitan derivatives such as polyethylene glycol(20) sorbitan monolaurate, polyethylene glycol(20) sorbitan monostearate.

Emulsifiers may also be advantageously selected from the group of the glyceryl derivatives of saturated and unsaturated fatty acids, in particular mono-, di-, tri- and polyglycerol derivatives including polyglyceryl diisostearate, polyglyceryl-2-oleyl ether, polyglyceryl-6-distearate, polyglyceryl-4-oleyl ether.

Furthermore, ethoxylated glyceryl esters may also be employed. As ethoxylated triglyceride polyethylene glycol (20) glyceryl tristearate may be used, for example. Another choice is to employ ethoxylated alkyl ethers such as polyethylene glycol dodecyl ether (Brij30) or polyethylene glycol hexadecyl ether (Brij52).

Furthermore, the emulsifiers may also be taken from the group of fatty alcohol (C₁₆-C₁₈) glucosides. Sucrose stearate, sucrose palmitate, Plantacare 1200 UP and Plantacare 2000 UP may beneficially be chosen as alkyl glucoside.

### Co-Emulsifiers

The co-emulsifiers of the microemulsions according to the invention are advantageously be taken from the group of phospholipids, for example:
Lecithin from plants (e.g. soybean, rapeseed, cottonseed) and egg yolk,
Phosphatidyl choline from soybean and egg yolk,
Phosphatidyl ethanol amine,
Phosphatidyl serin,
Phospatidyl inosite from soybean, rapeseed, cottonseed,
hydroxylated lecithin.

The co-emulsifier is beneficially selected from the group: Lecithin from soybean and egg yolk, known under the tradenames of Epikuron 135, Epikuron 170, Epikuron 200, Epikuron 200 SH, Phospholipon 25, NAT-8539 (Nattermann).

The amount of phospholipids (one or several compounds) in the preparations preferably ranges between 0.1 and 10% w/w, especially preferred between 0.5 and 5% w/w, in particular 1 and 5% w/w based on the total weight of the formulation.

Also suitable as co-emulsifiers are cholesterol and cholesterol derivatives: As ethoxylated cholesterol derivative polyethylene glycol (10) soybean sterol may beneficially be employed.

### Solvents

The following types of solvents may, for example, be used for the production of primary W/O microemulsions:
short- and long chain alcohols (e.g. ethanol, propanol, isopropanol),
Glycols (propylene glycol, 1,2-octane diol, 1,2-hexane diol),
Glycerol, digylcerol,
Pyrrolidones (N-methylpyrrolidone, N-ethyl pyrrolidone),
Carbohydrate derivatives (dimethyl isosorbide (Arlasolve®), Sorbitol).

The solvent is beneficially selected from the group: Ethanol, isopropanol, 1,2-octane diol, propylene glycol, N-methylpyrrolidone, dimethyl isosorbide.

### Active Substances

It is, moreover, possible and advantageous to use the inventive preparations as a basis for pharmaceutical formulations. It may be found difficult in this context to clearly differentiate between purely cosmetic and purely pharmaceutical products. According to the invention all substance classes are basically suited for use as pharmaceutically active agents, with lipophilic active substances are given preference. Examples are antihistamine drugs, antiphlogistics, antibiotics, antimycotics, virostatics, chemotherapeutics, perfusion-promoting substances, keratolytic agents, hormones, steroids, vitamins and the like.

Within the scope of the present invention it is considered beneficial to add to the preparations further anti-irritative or anti-inflammatory active substances, in particular NSAR (non-steroidal antirheumatics), corticosteroids, phytopharmaceuticals and extracts from plants.

Topical medical preparations within the meaning of the present invention as a rule contain one or several medicinal drugs of effective concentration.

In the event extracts from plants are used these are advantageously obtained from: Angelica root, arnica blossoms, basil leaves, birch, borage seed, chili, Curcuma longa, Curcuma xynthorrhiza, dill seed, peanut, fennel, clove, raspberry seed, hop, ginger, iris root, St John's wort, camomile blossoms, cardamom, carrot seed, Kava-Kava, lavender blossoms, marjoram leaves, lemon balm leaves, nutmeg, myrrh, oregano leaves, capsicum, peppermint leaves, marigold blossoms, rosemary, sage, sea buckthorn, star anise, fir, thyme, bourbon vanilla, frankincense, cinnamon. Especially advantageous is the use of angelica root, arnica blossoms, birch, borage seed, Curcuma longa, Curcuma xynthorrhiza, myrrh, fir, frankincense and cinnamon.

Fat-soluble vitamins are expediently taken from the group:

Vitamin A and derivatives, vitamin C (ascorbic acid) and derivatives, vitamin E (tocopherol) and derivatives. Tocopherol acetate and ascorbic acid palmitate may be used, for example. Ubichinone and its derivatives may as well be used.

Antioxidants are preferably taken from the group: Amino acids (e.g. glycine, histidine, tyrosine) and their derivatives, imidazoles, carotenoids, carotenes (e.g. α- and β-carotenes) and their derivatives, thiols (glutathione, thioredoxin, cysteine, cystamine and their derivatives), vitamin E and its derivatives. Of special advantage is tocopherol acetate.

The proportion of antioxidants (one or several compounds) in the preparations is preferably 0.001 to 30% w/w, especially preferred is 0.05 to 20% w/w, in particular 1 to 10% w/w in relation to the total weight of the preparation.

The inventive microemulsions may contain diffusion promoting agents such as N-methylpyrrolidon, dimethyl isosorbid, menthol, pinene, thymol, camphor, caffeine, diethylene glycol ester, e.g. diethylene glycol monoethyl ester, diethylene glycol. The diffusion promoting agents may advantageously be selected from the group of N-methylpyrrolidon, dimethyl isosorbide and diethylene glycol monoethyl ester.

The pharmaceutical products containing microemulsions according to the invention are beneficially selected from the group of antihistamine drugs, antiphlogistics, antibiotics, antimycotics, antipsoriatics, virostatics, chemotherapeutics, perfusion-promoting substances, keratolytic agents, hormones, steroids.

For the production of the primary W/O microemulsions the following types of essential oils have proven their worth: Terpenes (mono-, sesquiterpenes, diterpenes): citrus oil, pine, camomile; alcohols (monoterpenols, sesquiterpenols, diterpenols): ravensara, hyssop, niaouli; Aldehydes: lemon balm, eucalyptus; ketones (monoterpene ketones, sesqui- and diterpene ketones): yarrow, thuja as well as ester (monoterpene ester): lavender oil, ylang ylang; phenols: thyme; phenyl ethers: anise, clove; oxides: cineole; lactones: patchouli, frankincense and coumarins.

It is especially advantageous to select the essential oils from citrus oil, pine, camomile, ravensara, hyssop, niaouli, lemon balm, eucalyptus, yarrow, thuja, lavender oil, ylang ylang or tea tree oil.

The proportion of essential oils (one or several compounds) in the preparations preferably ranges between 0.1 and 10% w/w, especially preferred between 0.5 and 5% w/w, in particular between 1, and 5% w/w based on the total weight of the formulation.

Essential fatty acids are preferably taken from linoleic and γ-linolenic acids or essential fatty acid containing oils.

Of special advantage are γ-linolenic acid, borage oil, evening primrose oil and fish oil.

It was found that by the addition of water or aqueous solutions both secondary W/O and O/W microemulsions can be prepared from the primary W/O microemulsion. These are particularly suited to absorb water-soluble cosmetic and/or pharmaceutical substances. In the primary or secondary W/O microemulsion or O/W microemulsion the following water-soluble substances may be contained:

Pharmaceutical products may stem from antihistamine drugs, antiphlogistics, antibiotics, antimycotics, antipsoriatics, virostatics, chemotherapeutics, perfusion-promoting substances, keratolytic agents, hormones and steroids.

Also suited for use are antioxidants such as hydroxy- and dihydroxy benzoates, hippurates, salicylates, cysteine and derivatives, glutathione, vitamin C and its derivatives (e.g. Mg-ascorbyl phosphate, ascorbyl acetate) and vitamin H, superoxide dismutase, catalase, polyphenols, isoflavones. Of special benefit are ascorbyl acetate, superoxide dismutase, cysteine and glutathione.

Water-soluble vitamins and provitamins are selected from the group of vitamin B complexes, vitamin C and derivatives, vitamin H and derivatives, biotin, pantothenic acid, panthenol.

Furthermore, diffusion promoting agents may be employed such as terpenes, cineoles, menthol, propylene glycol, butylene glycol, polyethylene glycol with 4 to 250 ethylene glycol units, N-methylpyrrolidone, dimethyl isosorbide, diethylene glycol ester, e.g. diethylene glycol monoethyl ester, diethylene glycol, oleinic acid, acids or salicylic acid. Of special advantage are N-methylpyrrolidone, dimethyl isosorbide, salicylic acid, cineole, menthol, diethylene glycol monoethyl ester and oleinic acid.

Saccharides and oligosaccharides may beneficially be taken from the group: Glucose, fructose, mannose, mannitol, inositol, N-acetyl-D-glucosamine, D-glucosamine, chitooligosaccharides, trehalose. Of special advantage are glucose, D-glucosamine, N-acetyl-D-glucosamine, chitooligosaccharides and trehalose.

As polysaccharides chitosane, hyaluronic acid, heparin, dextran, cellulose ester or alginic acid may be incorporated into the preparations. Preference in this regard is given to chitosane and hyaluronic acid.

Moreover, proteins and protein derivatives from the group of the structural proteins may be used such as collagen, fibrin, elastine, with special preference being given to collagen.

As hormones or hormone-like substances for example hydrocortisone and its derivatives, melatonin, glycyrrhizinic acid and their derivatives as well as other plant-based hormones and plant-based steroids may be employed, with melatonin and glycyrrhizinic acid being preferred.

Extracts from plants may as well be used, in particular from the group: Meristem extract, aloe vera, echinacea, hamamelis extract, asparagus extract, neem tree, Polyplant microemulsion, horse chestnut, red grape leaves, arnica, marigold, ivy, nettles, camomile, horsetail.

Also suitable for use are amino acids, peptides, protein hydrolysate, e.g. silk protein hydrolysate, yeast hydrolysate, wheat protein hydrolysates. Preferred are silk protein hydrolysate and yeast hydrolysate.

### Additives

The inventive microemulsions may contain further additives. Included here are electrolytes, in particular of one or several salts with the following anions: chloride, sulfate, carbonate, phosphate. Advantageously, suitable for use are also electrolytes based on organic anions, for example lactates, acetates, benzoates, salicylates, propionates, tartrates, citrates and others. Especially preferred are potassium chloride, common salt, magnesium sulfate, zinc sulfate and mixtures thereof. Salt mixtures also considered beneficial are those occurring in the natural salt of the Dead Sea. Preferred for use as cations of the salts are ammonium ions, alkyl ammonium ions, alkali metal ions, alkaline earth metal ions, magnesium ions, iron ions and zinc ions.

The microemulsion according to the invention beneficially contains chelating substances (ethylene diamine tetraacetic acid or its salts, deferoxamine, histidine). Particularly advantageous is ethylene diamine tetraacetic acid (EDTA).

The inventive microemulsions advantageously contain humectant substances (NMF—natural moisturizing factor) (glycerin, ectoines, sorbitol, PCA-Na, urea, allantoin, glucosamine, chitosane, chitooligosaccharides, carboxylic acids, hydroxycarboxylic acids and dicarboxylic acids as well as polysaccharides, hyaluronic acid or aloe vera extract). Preferred are glycerin, urea, sorbitol, allantoin, PCA-Na, lactic acid, chitooligosaccharides, hyaluronic acid, chitosane, aloe vera extract.

The inventive microemulsions may also contain chemical oxygen carriers such as organic and inorganic peroxides, e.g. hydrogen peroxide, benzoyl peroxide.

Also suitable as additive substances are chemical and natural bleaching agents, e.g. hydroquinones, kojak acid, arbutin, acelainic acid, lemon juice and cucumber juice.

As pharmaceutically accepted oxidizing substance hydroquinone may, for example, be used.

Salicylates, benzoates, parabens, essential oils and plant-based extracts may, for example, be used as preservation substances.

The present invention is elucidated in sufficient detail by way of the following examples.

### EXAMPLE 1

### Primary W/O Type Honey Microemulsion

The primary W/O type honey microemulsion on the basis of synthetic oils was prepared as per the following formulation. Raw substances are given in % (w/w)

TABLE 1

| Constituents | 1 | 2 | 3 | 4 | 5 | 6 |
|---|---|---|---|---|---|---|
| Honey | 15 | 23.9 | 14.7 | 19.5 | 12.0 | |
| Polyglycerin- | | | 9.2 | 8.0 | | |
| 6-oleate | | | | | | |
| L.A.S | | | 17.1 | 16.0 | | 18.3 |
| (Gattefosse) | | | | | | |
| Span 80 | 8.0 | 11.4 | | | 10 | 9.2 |
| TWEEN 80 | 16.0 | | | | | |
| TWEEN 20 | | 22.7 | | | 22.0 | |
| Ethyl oleate | | 30.0 | | 28 | | |
| Isopropyl | 25.0 | | 29.0 | | | |
| myristate | | | | | | |
| Myritol | | | | | 27.0 | 25.0 |
| Phosphatidyl | 8.0 | 2.0 | 9.0 | | | |
| choline | | | | | | |
| NAT (Rhône- | | | | 10.5 | 11.0 | 12.0 |
| Poulenc) | | | | | | |
| Ethanol, 96% | 5.0 | | 5.0 | | 5.0 | |
| Propylene glycol | | | 5.0 | | 10 | 6.0 |
| Isopropanol | | 5.0 | | 3.0 | | 6.0 |
| Arlasolve ® | | | 6.0 | 7.0 | | |
| N-methyl | | 5.0 | | | 4.0 | |
| pyrrolidone | | | | | | |
| Water | 3.0 | 5.0 | 5.0 | 11.0 | 9.0 | 23.2 |

### Preparation of a Surfactant Mixture.

W/O and O/W emulsifier (e.g. 2 ml of Tween-80, 1 ml Span-80), 3 ml Isopropyl myristate and 0.6 g Phosphatidyl choline (89%) were mixed at room temperature.

### Preparation of a Basic Honey Microemulsion

2 ml of honey and 0.5 ml of distilled water were then added to the solution so obtained. The resultant turbid solution was stirred until a clear yellow solution of medium viscosity had developed. The particle size of the W/O microemulsion was found to be in the range of between 30 and 70 nm.

### EXAMPLE 2

### Preparation of a Primary W/O Honey Microemulsion Containing as Oil Phase Natural Oils or Mixtures Thereof

The W/O microemulsion was prepared similarly to the one described in example 1—No. 1 to 6 (Table 1), with exception that instead of IPM in the oil phase of the microemulsion oil mixtures based on natural oils (10 to 30%) were used either individually or as a mixture in IPM or ethyl oleate. The following natural oils were employed: Almond oil, olive oil, jojoba oil, avocado oil, cocoa butter, fish oil, borage oil, evening primrose oil, dogrose oil, linoleic and linolenic acid and the like.

The resultant clear W/O microemulsion showed a particle size ranging between 40 and 100 nm.

### EXAMPLE 3

### Preparation of a W/O Honey Microemulsion to which Oily Plant Extracts (inter alia CO₂ Extracts) were Added

To the oil phase 1% to 30% of oily and/or alcoholic extracts were admixed (comfrey, Curcuma longa, Curcuma xanthophyllis, horse chestnut, ginger). The honey microemulsion (ME) was prepared as described in example 1. In this way a clear honey microemulsion with particle sizes ranging between 60 and 110 nm was obtained.

Moreover, the following plant-based extracts were also used in a W/O honey microemulsion in the amounts indicated above, separately or as mixtures:

Echinacea purpurea (purple coneflower), Hypericum perforatum (St John's wort), Rosmarinus officinalis (rosemary), Salix sp. (willow), Passiflora incarnat (passion flower), Zarpagophytum procumbens (rampion), ginseng rad., Cisti icanin (rockrose), Betula fol. (birch), Vitis vinifera Fol (grapevines).

### EXAMPLE 4

### Preparation of an O/w Microemulsion Containing Essential Oils

As described in example 3, but instead of the CO₂— or alcoholic extracts essential oils (up to 15%) were admixed to the oil phase. The W/O microemulsions were prepared similarly to the one described in example 1—No. 1 (Table 1), with exception that instead of IPM oil mixtures based on essential oils (10%) were used in IPM or ethyl oleate. The following essential oils were put to use: Citrus oil, pine, lavender oil, ylang-ylang, camomile, ravensara, hyssop, niaouli, anise, clove, thyme, patchouli, frankincense, yarrow, thuja, lemon balm, eucalyptus, Rosa rubiginosa, Innophyllum callophyllum, linoleic and linolenic acid: borage oil, evening primrose oil, dogrose oil and also 1,4-cinneole (20%), menthol (20%) and limes (20%) in isopropyl myristate or ethyl oleate.

In this way clear to slightly opalescent honey microemulsions were obtained.

### EXAMPLE 5

### Preparation of a W/O Microemulsion Containing Fat-Soluble and Water-Soluble Vitamins and Provitamins

The following fat-soluble vitamins (vitamins E, A, D) were dissolved either separately or as mixtures in an oil phase in concentrations ranging between 0.01 and 10%. The primary W/O honey microemulsion was mixed by adding honey and water or aqueous vitamin-containing solutions in a manner as indicated in example 1 (Nos. 1 to 5). The particle sizes of the W/O microemulsion were found to be in the range of between 50 and 100 nm.

### EXAMPLE 6

### W/O Honey Microemulsion Containing Polysaccharides and Oligosaccharides

Using 0.1 to 10% aqueous chitooligosaccharide solutions (Oligopharm Inc., Russia, Korea) the primary W/O honey microemulsions indicated in example 1 (Nos. 1 to 5) were transformed into secondary W/O or O/W honey microemulsions. In this way a microemulsion of medium viscosity was obtained.

The primary W/O honey microemulsions were also prepared by adding aqueous oligosaccharide- and/or polysaccharide-containing solutions of series oligosaccharides (trehalose, fructo-, manno-, chitooligosaccharides), polysaccharides inter alia hyaluronic acid, heparin, chitosane, dextran, xanthan, hydroxypropyl cellulose and other cellulose derivatives, carrageenan, pectin, aloe vera.

### EXAMPLE 7

### Preparation of a W/O— and O/W Honey Microemulsion Containing Aqueous or Alcoholic Plant Extracts and (or) Microbiological Extracts

The W/O microemulsions were prepared in a manner similar to those described in example 1—Nos. 1 to 6 (Table 1). In lieu of water the following plant-based extracts or mixtures thereof were used: Coneflower (echinacea), green tea, hamamelis, neem trea, aloe vera, birch, horse chestnut, St John's wort, grapevines, willow, comfrey, dogrose, gingko, grapes, lime, mint, camomile, blueberry, raspberry etc.

The O/W honey microemulsions are prepared by adding one part of a primary W/O honey microemulsion (Nos. 1 to 6 of example 1) mixed with 2 parts of plant extracts. This resulted in the formation of a clear slightly opalescent O/W honey microemulsion with particle sizes ranging between 70 and 120 nm.

### EXAMPLE 8

### W/O and O/W Honey Microemulsions Containing Hormones or Hormone-Like Substances: Glucocorticoids, Mineralocorticoids, Androgens, Estrogens, Insulin, Calcitonin, Thyroxine, Prolactin, Somatotropin, Oxitocyn, Progesterone, Adrenaline, Erythropoetine, Phytosterols.

A 10% hydrocortisone solution in N-methylpyrrolidone was dissolved in an oil phase in concentrations of 0.01 to 3.0° A). The primary W/O honey microemulsions were mixed by adding honey and water or aqueous vitamin-containing solutions in a manner as indicated in example 1 (Nos. 1 to 5).

In this way a clear hydrocortisone-containing honey microemulsion was obtained with particle sizes ranging between 35 and 65 nm.

### EXAMPLE 9

### W/O Honey Microemulsion Containing Local Anesthetics

2.5 g of lidocaine base were added to the oil phase (No. 2, Table 1) and thoroughly stirred until the lidocaine had dissolved completely. Further preparation steps are similar to what has been described in example 2, table 1 with the exception that a 5% aqueous lidocaine×HCl solution was used. In this way a clear honey microemulsion of medium viscosity was obtained with particle sizes ranging between 60 and 110 nm. Microemulsions with prilocaine, procaine, benzocaine, morphine, codeine, dihydrocodeine, methadone, clofenadone or pentazocine can be prepared in a similar manner.

### EXAMPLE 10

### O/W Diclofenac Honey Microemulsion

Instead of propylene glycol (example 5, table 1) 10% diclofenac Na in propylene glycol was added to an oil/surfactant mixture. Further preparation steps are carried out as per example 5 (table 1). In this way a clear honey diclofenac microemulsion was obtained with particle sizes ranging between 90 and 150 nm. In an analog manner microemulsions can be prepared with indometacin, ibuprofen, ketoprofen, piroxicam, acetylsalicylic acid or metatrexate.

### EXAMPLE 11

### W/O Honey Microemulsion with Active Ingredients for a Photodynamic Therapy

The W/O honey microemulsions were prepared similar to the description in example 1—Nos. 1 to 6 (table 1), however instead of water 0.01 to 1.0% aqueous solutions of the following photosensitizers were used: 5-aminolevulinic acid, methyl-5-amino-4-oxopentanoate (MAOP) and other derivatives of 5-aminolevulinic acid, porphyrins, inter alia hematophorphyrine (Photofrin®), Photoporphyrin IX (PP9), chlorin, phtalocyanides (e.g. sulphophthalocyanine—Phtalosens-Lio®).

### EXAMPLE 12

### W/O Honey Microemulsions with Antibacterial and/or Antifungal Active Substances

In lieu of propylene glycol (example 5, table 1) 10% ciclopiroxamine in ethanol was added to an oil/surfactant mixture. Further preparation steps are carried out as per example 5 (table 1). In this way a clear cyclopirox honey microemulsion was obtained with particle sizes ranging between 90 and 130 nm. Microemulsions with terbinafine, clotrimazole, nystatin etc. can be prepared in a similar way.

### EXAMPLE 13

### Physical Properties of a Secondary W/O and an O/W Honey Microemulsion

Honey microemulsion No. 3 (table 1) was thoroughly mixed with different amounts of water. This resulted in clear transparent or slightly opalescent secondary W/O or O/W honey microemulsions (table 2) being produced.

TABLE 2
Physical properties of the W/O and O/W honey microemulsions

| | 7 | 8 | 9 | 10 | 11 |
|---|---|---|---|---|---|
| Honey microemulsion, | 3.0 | 3.0 | 3.0 | 3.0 | 3.0 |
| ml | | | | | |
| Water, ml | 1.0 | 3.0 | 4.0 | 6.0 | 12.0 |
| Visual appearance | Clear ME | Clear ME | Clear ME | Clear ME | Clear ME |
| Dynamic viscosity¹⁾, | 120 ± 13 | 70 ± 9.5 | 40 ± 2.4 | 24 ± 2.1 | 18 ± 1.5 |
| mPa · s | | | | | |
| Osmolarity²⁾, (mos- | 320.0 ± 0.26 | 310.0 ± 0.18 | 290 ± 0.19 | 280 ± 0.25 | 280 ± 0.3 |
| mol/kg) | | | | | |
| Particle size³⁾, nm, | 50 ± 5 | 76 ± 6.7 | 98 ± 8.7 | 103 ± 11.0 | 112 ± 10.6 |

¹⁾Rotational viscometer Fluids spectrometer RFS II (Rheometric Scientific), shear rate 0.1 to 100 (1/s)
²⁾Colloid Osmometer (Knauer)
³⁾Dynamic light scattering (Zetasizer NanoZS90, Malvern)

### EXAMPLE 14

### Demonstration of the Penetration Depth of Honey Microemulsions into the Skin

Immediately after slaughtering pig ears without skin injuries were picked up from a slaughterhouse. The ears were thoroughly washed with mild soap. Immediately afterwards, the subcutaneous layer of fat was removed using a scalpel. The skin was stored in a container at −20° C. for further examination. Separating the epidermis from the dermis was carefully carried out after heating at 60° C. (1 minute).

The penetration tests were carried out with the help of a Franz diffusion cell which had an effective diffusion area of 0.75 cm² (Crown Glass Co, Inc.).

1 ml of microemulsion was introduced into the donor compartment of the cell. An isotonic NaCl solution was used as acceptor fluid. After a defined period of time an aliquot (0.5 ml) was taken and supplemented with an equal amount of PBS buffer.

The penetration depth of microemulsions was assessed on a model skin in the framework of a peeling process. The microemulsions (example 10) were applied to the ear skin surface and massaged into the skin for about 1 to 2 minutes while the temperature of the ears was kept at 35° C.

For the peeling process 15 adhesive tape (Tesafilm) strips (type Tesa® Office, Beiersdorf, Del.) were cut to a size of 1.5 cm*1.5 cm and weighed. The prepared adhesive tape strips were successively stuck on the examination areas 2 cm*2 cm in size selected on the pig ear and afterwards peeled off.

The strips were leached with acetonitrile at 60° C. in the course of 60 minutes. The solutions were filtered through 0.5 μm membrane and analyzed with the aid of HPLC. The Shimadzu LC-20A HPLC system consisted of a Prevail™ carbohydrate ESHPLC column and Nucleosil 100-5 C₁₈ column, evaporative light scattering detector Sedex 75 (Sedere, France).

TABLE 3
Average retrieval values of 4 treated pig skins of the honey
microemulsions (example 10) in the individual skin layers after
4 hours of application

| Distribution of | Total sugar, μg/cm² | Diclofenac, μg/cm² |
|---|---|---|
| Corneal layer | 5.2 | 12.3 |
| Epidermis | 30.4 | 6.1 |
| Dermis | 21.3 | 5.0 |

As is evident, the honey microemulsions according to the invention enable a significantly enhanced penetration into the deeper skin layers so that active ingredients can act better and more effectively.

### EXAMPLE 15

### Demonstration of the Effectiveness of Cosmetic Treatment with the W/O Honey Microemulsion

The epicutaneous test with W/O honey microemulsion (example 3) performed on 40 female probands of all skin types, of which 13 were subjects with skin allergies, has shown that no undesirable reactions have occurred.

The cosmetic treatment with W/O honey microemulsion (example 3) took place over a period of 6 weeks involving a total of 15 voluntary female test persons aged between 45 and 65 years with dry and aged skin. Honey microemulsion (0.5 ml) was applied to forehead and the area of the mouth corners by means of an oxygen spraying device (Medprop Technology AG, St. Gallen, Switzerland), slightly massaged into the skin and aerated with 90% oxygen within a time period of 15 minutes. The treatment was performed twice a week. The test results of all female test subjects were assessed by means of the SELS process in the examination room. The evaluation measurements focused on the forehead and is the mouth corner area. With the aid of an optical 3D measuring device (Courage & Khazaka, Cologne) and applying the so-called SELS process the skin parameters as well as scruffiness, roughness, wrinkling and smoothness were determined and assessed. A skin smoothing effect of honey microemulsions was detected with 14 female test subjects (table 4).

TABLE 4
Skin smoothness parameters prior to and after treatment with honey
microemulsion (in SELS-SE units)

Skin area

| | |
|---|---|
| Forehead | Corner of mouth |

Roughness

| | | |
|---|---|---|
| Before | 0.6 ± 0.08 | 0.7 ± 0.09 |
| After | 0.5 ± 0.10 | 0.4 ± 0.14 |

Scurfiness

| | | |
|---|---|---|
| Before | 0.9 ± 0.17 | 0.85 ± 0.15 |
| After | 0.55 ± 0.12 | 0.6 ± 0.13 |

Smoothness

| | | |
|---|---|---|
| Before | 6.5 ± 1.0 | 4.4 ± 0.9 |
| After | 8.3 ± 1.1 | 7.0 ± 1.1 |

Wrinkling

| | | |
|---|---|---|
| Before | 8.1 ± 1.0 | 9.2 ± 1.2 |
| After | 7.1 ± 0.9 | 7.3 ± 0.8 |

### EXAMPLE 16

### O/W honey Microemulsion for the Skin Treatment of Cellulitis

One part of a primary W/O microemulsion from example 3 was mixed with 3 parts of an aqueous 0.1% solution of theobromine, nicotinic acid and caffeine such that a clear honey microemulsion was produced having particle sizes rangy ing between 60 and 130 nm.

18 female subjects took part in a three to five-week treatment course. The treatment was carried out three times a week and focused on the outer side of one thigh. The opposite side was left untreated and served control purposes. The elasticity of the skin was measured by a “Derma-Lab-Systems” cytometer.

TABLE 5
Relative increase in skin elasticity (ΔE/Eo) with 18 female subjects after
treatment periods of 3 and 5 weeks in comparison to untreated skin areas.

| Treatment period | Treatment | Skin elasticity, ΔE/Eo, % |
|---|---|---|
| 3 weeks | No | 2.1 ± 0.3 |
| | Yes | 32.3 ± 0.5 |
| 5 weeks | No | 2.7 ± 0.2 |
| | Yes | 41.1 ± 0.6 |

After a treatment time of 5 weeks the skin elasticity was found to have increased by 40%.

### EXAMPLE 17

### W/O and O/W Honey Microemulsions Containing Plant-Based Anti-Inflammatory Substances

The treatment of patients suffering from diseases with inflammatory background was carried out in a medical rheumatology office during the winter months of 2007. All treatment activities were performed by applying W/O honey microemulsion (example 3). The microemulsion in an amount of 0.3 to 0.5 ml was applied to the affected area using a spray device (oxygen generator, Medprop Technology AG, St. Gallen, Switzerland) and then gently massaged into the skin. Following this, the affected location was additionally aerated with oxygen gas (oxygen generator, Medprop Technology AG, St. Gallen, Switzerland) within a time span of 20 minutes. Treatment was as a rule carried out twice or three times a week. Success of the treatment was determined with the aid of the visual analog scale (VAS) and rated as a decrease in the relevant pain.

TABLE 6
Indications, number of cases and treatment success

| | Number | | |
|---|---|---|---|
| Indication | of patients | Success | No success |
| Arthritis of upper subtalar joint | 8 | 7 | 1 |
| Arthritis of knee joint | 6 | 4 | 2 |
| Arthritis of the hip | 7 | 6 | 1 |
| Pain in the wrist joint | 4 | 3 | 1 |
| Cervical spine syndrome | 5 | 4 | 1 |
| Vertebral column syndrome | 9 | 6 | 3 |
| Lumbar spine syndrome | 12 | 7 | 5 |
| Total | 51 | 37 | 14 |

As can be seen from the table the treatment success was 67° A).

### EXAMPLE 18

### Pain Relieving Honey Microemulsion

The effect of the pain relieving honey microemulsion (of example 9) was tested with 12 voluntary test persons (4 male and 8 female, aged between 22 and 35 years). The honey microemulsion was examined in comparison to the EMLA® cream (AstraZeneca). Approx. 2 g of EMLA® cream and 0.4 ml of honey microemulsion were applied to 10 cm² of skin. After different exposure periods the sensation of pain was checked through little pinpricks. The participating persons assessed their pain sensation on a pain intensity scale (O— no pain, 5—normal pain and 10—intense pain).

TABLE 7
Time-analgesic potential of the honey microemulsion and EMLA ®
cream

| Vehicle | Time, min. | Pain intensity |
|---|---|---|
| Honey microemulsion | 15 | 8.0 |
| | 30 | 6.5 |
| | 45 | 2.0 |
| | 60 | 2.0 |
| EMLA ® cream | 15 | 9.5 |
| | 30 | 9.0 |
| | 45 | 6.0 |
| | 60 | 3.0 |

The honey microemulsion showed moderate analgesic effects after 30 minutes and effective analgesic effects after 45 minutes whereas EMLA® cream showed effects after only 1 hour.

### EXAMPLE 19

### Therapy of Horses Suffering from Inflammatory Diseases

All treatment activities were performed by applying W/O honey microemulsion (example 3). The microemulsion in an amount of 0.5 to 1.0 ml was applied to the affected area using a spray device (oxygen generator, Medprop Technology AG, St. Gallen, Switzerland). Following this, the affected location was additionally aerated with oxygen gas (oxygen generator, Medprop Technology AG, St. Gallen, Switzerland) within a time span of 20 minutes. Treatment was as a rule carried out twice or three times a week.

TABLE 8
Therapy of 84 horses suffering from inflammatory diseases

| | Disease | | | No | |
|---|---|---|---|---|---|
| Indication | cases | Success | | success | |
| Coffin joint inflammation | 12 | 10 | | 2 | |
| Fetlock joint inflammation | 19 | 18 | | 1 | |
| Sesamoidosis disease | 27 | 25 | | 2 | |
| Tarsal inflammation of the | 3 | 3 | | 0 | |
| forefoot | | | | | |
| Suspensory ligament | 5 | 5 | | 0 | |
| damage | | | | | |
| Chronic phlegmons | 2 | 2 | | 0 | |
| Subtalar joint arthrosis | 8 | 6 | | 2 | |
| Subtalar joint inflammation | 8 | 8 | | 0 | |
| Total (in %) | 84 | 77 | (92) | 7 | (8) |

As can be seen from the table the treatment success was 92%.

### EXAMPLE 20

### Milk Beverage Based on a Chitooligosaccharide Honey Microemulsion

The antibacterial effect of the microemulsion of 0.5 to 5% concentration in milk was tested with respect to gram-positive bacteria (Listeria monocytogenes) and gram-negative bacteria (Escherichia coli 157:H7) (example 6).

Honey microemulsion (2%) in milk (3.5% fat content) was found to completely inhibit the growth of Listeria and E. coli within a period of seven days.

50 test persons liked the taste of the milk product, 6 subjects had a negative opinion about it.

### EXAMPLE 21

### Dietary Supplement Based on Chitooligosaccharide-/Natural Juice Honey Microemulsions

The honey microemulsion described in example 11 was sealed into a gelatine capsule (250 μl microemulsion per capsule).

The product (example 6) contains chitooligosaccharides (COS), natural juice of pumpkins, carrots and Jerusalem artichokes. Aside from honey this complex provides valuable substances aiding the prophylaxis of functional disorders of the liver. Chitooligosaccharides are known to bond and remove toxic substances. Moreover, COS themselves efficiently stimulate and regulate the lever cell functions.

The effectiveness of the honey microemulsion (example 6) with patients suffering from liver function disorders as well as the accelerated restoration of damaged hepatocytes were demonstrated in a clinical study undertaken during a period from Jan. 24, 2007 to Mar. 1, 2007.

Based on medical history involving frequently recurring alcohol abuse, occurrence of a cytolysis phenomenon, high gamma glutamate transpeptidase (GGTP) and alanine aminotransferase (ALT) values, enlargement of liver and spleen, and icterus 22 patients aged between 24 and 40 years were diagnosed with alcoholic hepatitis. The verum group comprised 12 male patients while the control group consisted of 10 patients showing a similar clinical and laboratory history of a liver disease. The patients were prescribed 2 tablets to be taken twice a day over a period of one month.

6 of the patients were diagnosed with a toxic-allergical (medicational) hepatitis. Their medical history showed that shortly before they fell ill they had taken preparations that contained sulfonamides, antipyretic febrifugal and antimycotic medicaments. In these cases fever, dyspeptic indications, skin itching of varying intensity and pustular skin rash were detected as clinical symptoms.

The study showed the following results:
the elimination of dyspeptic indications began 5-6 days earlier with patients who took the honey microemulsion preparation;
in the verum group the bilirubin anemia normalized 6-8 days earlier;
with all patients in the verum group the puritus disappeared on the 14^{th} to 16^{th} day of the therapy, in the control group on the 15^{th} to 20^{th} day. Only 2 patients suffered from puritus for more than 30 days.

TABLE 9
Average biochemical blood parameters of the patients prior to and after
treatment with honey microemulsion}

| Meas- | | | | | |
|---|---|---|---|---|---|
| ured | | | | | |
| param- | Normal | Verum group | | Control group | |
| eters | values | before | after | before | after |
| Bili- | 8.0 | 164 ± 12 | 35 ± 9 | 158 ± 11 | 121 ± 13 |
| rubin | | | | | |
| ALT | 0.75 | 3.8 ± 0.8 | 1.2 ± 0.6 | 4.0 ± 0.6 | 2.7 ± 0.8 |
| GGTP | 400 | 2680 ± 18 | 700 ± 20 | 2800 ± 20 | 1350 ± 25 |

ALT—Alanine aminotransferase,
GGTP—Gamma glutamate transpeptidase

The positive effects of the product are that it is capable of stopping both exogenous and endogenous intoxications and thus proactively counteract liver damage caused by alcohol and medical preparations. The product rich in COS, flavonoids and carotenoids enables a protection of the hepatocyte membrane.

1. Microemulsion containing:
50 to 80% w/w of an oil phase,
2 to 40% w/w of a mixture of one or several W/O emulsifiers and one or several O/W emulsifiers at a ratio of 1:5 to 1:1,
5 to 40% w/w of honey, royal jelly, propolis and/or perga
0.01 to 30% w/w of co-emulsifiers and
1 to 20% w/w of water or an aqueous solution.

2. Microemulsion according to claim 1, characterized in that the oil phase is formed by alkane carboxylic acid ester, dialkyl ether, alcohols, fatty acid triglycerides and/or low-volatile hydrocarbons.

3. Microemulsion according to claim 1, characterized in that the microemulsion contains 0.01 to 25% w/w of water-soluble and/or fat-soluble solvents.

4. Microemulsion according claim 3, characterized in that the solvent is selected from the group of mono- and polyvalent alcohols, polyols, pyrrolidones, carbohydrate derivatives.

5. Microemulsion according to claim 1, characterized in that the W/O and O/W emulsifiers are non-ionic and the W/O emulsifiers have an HLB value of between 2 and 7 and the O/W emulsifiers have an HLB value ranging between 9 and 18.

6. Microemulsion according to claim 5, characterized in that the W/O and O/W emulsifiers are selected from the group: ethoxylated fatty alcohols, sorbitan derivatives, ethoxylated sorbitan derivatives, glyceryl derivatives of saturated or unsaturated fatty acids such as mono-, di-, tri- and polyglycerol derivatives, ethoxylated glyceryl ester, ethoxylated alkyl ether, fatty alcohol (C₁₆-C₁₈) glucosides.

7. Microemulsion according to claim 1, characterized in that the co-emulsifiers stem from the group of phospholipids.

8. Microemulsion according claim 7, characterized in that the phospholipids are selected from the group: Lecithins, phosphatidyl ethanol amine, phosphatidyl serin, phosphatidylinositol, hydroxylated lecithin.

9. Microemulsion according to claim 1, characterized in that the microemulsion contains 0.01 to 30% w/w, in particular 1 to 20% w/w of one or several water-soluble or fat-soluble active ingredients.

10. Microemulsion according claim 9, characterized in that the active ingredients are selected from the group: Analgesics, local anesthetics, antiphlogistics (antirheumatics), glucocorticoids, antibiotics, antimycotics, virustatics, immunosuppressive agents, immunomodulators, antipsoriatics, keratolytic agents, hormones, phytopharmaceuticals, lipophilic plant-based extracts, flavonoids, isoflavones, xanthophylls, polyphenols, alkaloids, glycosides, carotenoids, terpenes, terpenoids, phenolic acid derivatives, phytosterols, vitamins, provitamins, vitaminoids, antioxidants, hormones, saponines, essential oils, scents, unsaturated fatty acids, ceramides, phenolcarboxylic acid, cumarins, lignans, tannins, anthropoids, mono- and sesquiterpenes, triterpenes, sterols, flavoring substances, pharmaceutical substances, AHA acids, plant-based extracts, poly- and oligosaccharides, amino acids, proteins, protein hydrolysates, electrolytes, mineral nutrients, oxidants, chelating substances, diffusion promoting agents, humectants.

11. Secondary W/O microemulsion or O/W microemulsion consisting of a mixture of a microemulsion claim 1 and water or an aqueous solution at a ratio of 1:1 to 1:100.

12. Use of a microemulsion according to claim 1 as medical, veterinary or cosmetic preparation for topical, percutaneous or peroral administration.

13. Use of a microemulsion according to claim 1 for skin care, treatment of skin diseases, skin aging symptoms and/or moisture regulation of the skin.

14. Use of a microemulsion according to claim 1 for the production of foodstuff, dietary supplements or dietetic products.
